(19) United States
(12) Patent Application Publication
(10) Pub. No.: US 2024/0170158 A1
(43) Pub. Date: May 23, 2024

(54) METHOD FOR MANAGING A VIRTUAL PATIENT MODEL, PATIENT MODEL MANAGEMENT FACILITY, COMPUTER PROGRAM AND ELECTRONICALLY READABLE DATA CARRIER

(71) Applicant: Siemens Healthcare GmbH, Erlangen (DE)

(72) Inventors: Annette Birkhold, Stuttgart (DE); Ramarao Gundlapalli, South Barrington, IL (US); Michael Wiets, Langensendelbach (DE); Markus Kowarschik, Erlangen (DE)

(21) Appl. No.: 18/509,724

(22) Filed: Nov. 15, 2023

(30) Foreign Application Priority Data
Nov. 18, 2022 (DE) 10 2022 212 366.2

(51) Int. Cl. G16H 50/50; G06T 17/00; G16H 20/40

(52) U.S. Cl. CPC G16H 50/50; G06T 17/00; G16H 20/40

(57) ABSTRACT

A method for managing a virtual patient model of a patient for a series of treatment and/or examination procedures occurring over time, wherein at least at a first time point at which the patient is described by first patient parameters, the patient model describes at least the surface of the patient at the first time point, wherein at least at a second later time point at which the patient is described by second patient parameters, the patient model is extended by at least one model transformation using the first and second patient parameters for describing at least the surface of the patient at the second time point.

### CROSS REFERENCE TO RELATED APPLICATIONS

This application claims the benefit of DE 102022212366.2 filed on Nov. 18, 2022, which is hereby incorporated by reference in its entirety.

### FIELD

Embodiments relate to a method for managing a virtual patient model of a patient for a series of treatment and/or examination procedures occurring over time. At least at a first time point at which the patient is described by first patient parameters, the patient model describes at least the surface of the patient at the first time point.

### BACKGROUND

Virtual electronic patient models, that may also be referred to as “digital twins”, are used for many different purposes in the field of medical technology. For example, virtual patient models are used to improve X-ray dose management, to simplify the use of technical medical equipment by automating and accelerating imaging and treatment workflows and/or to provide navigation aids, for example for robotic systems and/or other systems, for example for minimally invasive interventions.

Virtual patient models may, for example, be generated from image data of the patient, wherein approaches have already been proposed in which an instance of a universal model is generated as a virtual patient model on the basis of patient parameters. For example, DE 10 2019 203 192 A1 relates to the generation of a digital twin for medical examinations. Here, it is for example also possible to adapt the virtual patient model to a body posture of the patient.

In medical technology, treatments and/or therapies are known that may extend over lengthy periods of time, for example several months or even several years. In such cases, treatment or examination procedures are performed at various times, for example at regular intervals. For example, in radiation therapy, it is known to administer radiation doses over time, i.e., at different times, to a specific body region. While, for many patients, for example adult patients, the same virtual patient model may be retained without major problems for the entire period of the treatment and/or examination, for example in order to enter examination results and/or treatment progress, problems always arise when there are major relevant changes to the patient's body over time. While this may, for example, occur in the case of weight changes in adult patients, for example in oncology, problems of this kind occur to a large extent in pediatric patients, i.e., people who are still growing. The growth of the patient causes structural changes to the body which hinder the use of the same or a single virtual patient model over the entire period of the examination and/or therapy. For example, it may be extremely difficult to identify the overlap of anatomical areas being treated or examined between an earlier time point and a later time point. In the context of radiation treatment, it is also necessary to consider that the stochastic risk of cancer may be estimated as higher for pediatric patients and therefore accurate tracking of an administered radiation dose over time is an important tool for evaluating risk.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art.

Embodiments provide an improved aid for tracking treatments and/or examinations extending over lengthy overall periods, that may for example be used when the patient's body changes over time.

Embodiments provide a patient model management facility, a computer program and an electronically readable data carrier.

Embodiments provide a method that, at least at a second later time point at which the patient is described by second patient parameters, the patient model is extended using the first and second patient parameters for describing at least the surface of the patient by at least one model transformation at the second time point.

Here, for example at the second time point, the patient's body has changed compared to the first time point, for example due to growth, for example in the case of a pediatric patient, and/or due to a change in weight, that may be due to illness, for example. The period between the first and the second time point may therefore be, for example, at least one month, for example at least one year.

A patient model is provided that is extended for an examination and/or treatment with several successive treatment and/or examination procedures over time, for example over an overall period, from a three-dimensional description of at least the surface of the patient (partial model) to four dimensions, i.e., the spatial dimensions and the time, by also deriving a description of at least the surface of the patient by a model transformation for each time point at which a treatment and/or examination procedure takes place and/or that is otherwise of interest, for example as an additional (three-dimensional) partial model. Since for example fixed predetermined model transformation is used, it is possible to derive a unique assignment for different locations on and/or in the patient, that may advantageously be used for tracking, as will be discussed in more detail below. Herein, it should be noted that the first time point does not necessarily have to be the time point of the creation of the virtual patient model. Rather, it is also possible for a second time point after the extension of the virtual patient model to become a new first time point for a subsequent extension, i.e., a later time point that then forms the second time point. The model transformation may be applied with reference to the time point of the creation of the virtual patient model as the first time point. Since the model transformation is for example predetermined, i.e., it cannot be changed for an examination and/or treatment over the overall period, in principle, these paths all lead to the same result.

Within the meaning of the first three-dimensional partial model, the patient model may preferably be created using image data of the patient. The patient model may also or alternatively be generated as an instance of a universal model that is created using the patient parameters or also further patient parameters. Although, for the extension of the virtual patient model to a later second time point, it is in principle conceivable to include image data that is taken into account during the model transformation, this is less preferable. Therefore, it is advantageous for the model transformation to be formulated independently of image data of the patient, for example image data from the second time point, and to depend solely on the first and second patient parameters, that are expediently easy-to-determine characteristics of the patient.

The patient parameters may for example include a height and/or weight and/or age and/or gender of the patient. Here, for purposes of simplification, it may be assumed that the gender of the patient will not change over the overall period of the examination and/or treatment. A change may also be taken into account in the model transformation. While, when applied to adults, it is sometimes sufficient only to consider weight as a patient parameter, when applied to pediatric patients who are still growing, it is been found to be advantageous to use at least height and/or weight as patient parameters. A further improvement in accuracy results when the patient's age is also used as a patient parameter. Age has been found to be useful because skeletal maturity is highly correlated with age.

If model transformation is used that, for example alone, depends on the easily identifiable patient parameters, i.e., for example their changes, this provides an uncomplicated easy-to-implement way of estimating the changes in the patient's body that have occurred compared to the first time point. For this purpose, only the model transformation parameterized via the patient parameters has to be applied to the three-dimensional description of at least the surface of the patient at the first time point, i.e., to the partial model at the first time point.

For example, therefore, embodiments provide for the creation of a four-dimensional patient model in terms of spatial coordinates and time. This allows tracking of locations in the patient model and hence correct assignment in the case of a plurality of examination and/or treatment procedures over an overall period. Hence, an easy-to-use useful tool is provided for correctly tracking influences, outcomes and/or results in treatment and/or examination over the overall period and/or sub-periods thereof.

Specifically, it may be provided that the patient model, for example at least for the surface of the patient, is at least a polygonal model with vertices and faces defined thereby, wherein the model transformation changes the position of the vertices in dependence on the first and second patient parameters. For example, a surface of the patient may be mapped by a polygonal network of this kind as what is known as a “mesh”. For example, here, in each case three vertices may be understood as spanning a part of the surface. The transformation changes the position of vertices, that also results in changes to the faces defined thereby, but no new vertices are added or removed and for example no neighborhood relationships are changed. In other words, this means that at any time point there is a unique assignment of the vertices at different time points as well as of the faces defined thereby at different time points. The vertices and hence the faces may be tracked over time since their identity does not change.

Spatial features of the patient model described by location information at the first time point may be tracked at the second time point. Herein, the location information may for example describe positions and/or regions of the virtual patient model (and hence also of the patient), for example vertices and/or faces of the polygonal model. In other words, it is always known where the location described by location information is located at the different time points since the model transformation establishes the corresponding unique relationship or does not change the identity. Therefore, if there is an influence on a location on a patient, i.e., an impact takes place at the first time point, despite the change in the body, the four-dimensional virtual patient model still enables it to be identified at the second time point where the corresponding location at which the impact took place is now located.

For example, for at least one time point, the location information may be assigned impact information relating to the treatment and/or examination procedures for creating a four-dimensional impact map based on the virtual patient model. In this context, it may be expedient for each feature or each feature of at least one class of features in the virtual patient model to be already assigned impact information that may be filled accordingly for the different time points at which it differs from a default value. For example, an impact at a specific location or a specific spatial feature may be quantified and entered directly into the patient model for the corresponding time point and, if applicable, subsequent time points as impact information, that is, for example, incremented from zero. When using vertices and faces defined thereby, this assignment may be made either to vertices or to faces, or if applicable also to both classes of spatial features. For example, in the case of assignment to vertices, information for the faces defined thereby may also be derived therefrom, for example by interpolation and/or extrapolation.

In an embodiment, in the case of radiation treatment and/or a radiation examination, the impact information may include dose information. Therefore, a four-dimensional dose map, that is important with pediatric patients for assessing risks and/or planning subsequent examination and/or treatment procedures, may be estimated, for example a skin dose map when describing the patient's surface. Adaptation of the patient model as the patient's body changes over time, for example the provision of corresponding partial models, and the use of model transformation provides locations or spatial features, for example vertices and/or faces defined thereby, on which radiation has acted to be tracked over time and impacts may also be summed up accordingly. Therefore, the use of the virtual patient model and model transformation provides the spatial changes of irradiated spatial features to be estimated and tracked over time. For example, in the case of a pediatric patient, it is possible to estimate that regions, for example skin regions, were irradiated at which time points with which radiation doses, for example X-ray doses, based on the concomitantly growing virtual patient model. For patients on whom a plurality of radiological procedures take place during growth, a four-dimensional irradiation map or dose map of the applied radiation, for example X-rays, is created. For example, when the surface of the patient is mapped by vertices and faces defined thereby, i.e., as a mesh, each vertex and/or each face may be assigned the dose entered there as a function of time, specifically as impact information. This approach may be applied not only to the growth of pediatric patients—it may also be used with adult patients whose body shape changes over time.

Embodiments may provide that a representation of the patient, for example at least of the patient's surface, is generated from the patient model for at least one of the time points covered thereby in which the impact information is reproduced true to location, for example by color coding. For example, this makes it possible to generate a time series of representations that visualize changes in both the body and the impact over time. Here, the use of model transformation means that a correct spatial assignment of even past impacts is always possible. In the case of a radiation dose, for example X-ray dose, in which the impact information forms dose information, color coding may, for example, take place depending on the level of the radiation dose entered.

It may be provided that model transformation is ascertained statistically and/or by machine learning. Herein, it has been shown that statistical evaluations are sufficient to permit excellent estimates. For example, it may be specifically provided that, for the statistical ascertainment of the model transformation, three-dimensional scan datasets of a population to be ascertained, that are provided and describe at least the surface of person for different time points and to which in each case the patient parameters for the respective time points are assigned, are subjected to principal component analysis (PCA) for classes of comparable patient parameters and/or at least substantially the same patient parameters and, to ascertain the model transformation, the results of the principal component analysis, that are for example brought into a modeling form corresponding to the patient model, are related to the assigned patient parameters. For example, therefore, it is possible to use scan datasets for a population to be ascertained recorded at different time points, i.e., with different patient parameters, that may entail the measurement of surfaces, for example with a 3D camera and/or a radar sensor, or also of the body as a whole, for example with a CT dataset. Scan datasets to which comparable patient parameters are assigned may be subjected to principal component analysis in order to extract the predominant characteristics that they have in common. Based on the results of the principal component analysis, a shape adjustment may then be performed, for example mapping as a polygonal model/mesh for the patient's surface. Incidentally, a corresponding description may also be applied to internal organs, if included. When the comparable patient parameters have been assigned to the results of the principal component analysis in each case, they may be related to one another in order to ascertain the model transformation in a known manner.

Embodiments provide a patient model management facility for managing a virtual patient model of a patient for a series of treatment and/or examination procedures occurring over time, wherein, at least at a first time point at which the patient is described by first patient parameters, the patient model describes at least the surface of the patient at the first time point. The patient model management facility includes an extension unit for temporally extending the patient model for describing at least the surface of the patient at a second time point at which the patient is described by second patient parameters by at least one model transformation using the first and second patient parameters. All statements relating to the method may be transferred analogously to the patient model management facility with which, therefore, the aforementioned advantages may also be obtained.

The patient model management facility may include a computing facility, that may, for example, have at least one processor and at least one storage. The extension unit may be one of a plurality of functional units implemented by hardware and/or software. Further functional units of the patient model management facility may, for example, include a creation unit for creating the virtual patient model, for example, therefore, the first partial model, and/or a representation unit for generating representations, for example of the impact information as described above. Interfaces, for example for receiving patient parameters, and/or input and/or output, for example for receiving patient parameters and/or for outputting representations, may also be provided. The patient model management facility may be embedded in an information system, for example a hospital information system and/or a radiology information system. It is for example possible for current or past patient parameters to be retrieved therefrom.

A computer program may be loaded directly into a storage a computing facility and has program code for performing the steps of a method when the computer program is executed on the computing facility. The computer program may be stored on an electronically readable data carrier and therefore includes control information stored thereon, that includes at least one computer program and is configured to perform the method when the data carrier is used in a computing facility.

### BRIEF DESCRIPTION OF THE FIGURES

FIG. 1 depicts a flow chart of an embodiment of the method.

FIG. 2 depicts a sketch explaining the method according to an embodiment.

FIG. 3 depicts a sketch explaining a four-dimensional patient model according to an embodiment.

FIG. 4 depicts representations of a four-dimensional impact map as a time series according to an embodiment.

FIG. 5 depicts the functional structure of a patient model management facility according to an embodiment.

### DETAILED DESCRIPTION

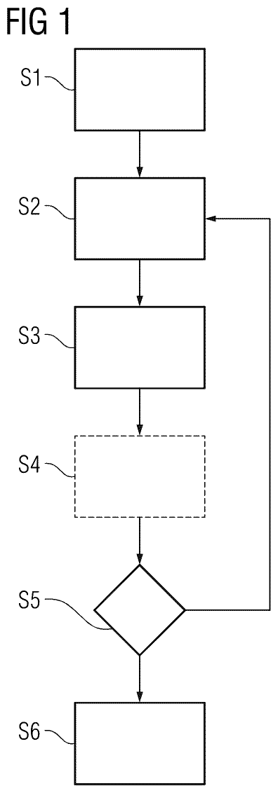

FIG. 1 depicts a flow chart of an embodiment of the method. In the present case, radiotherapy, for example with X-rays, that has taken place over several years in spaced-apart treatment procedures on a pediatric patient is to be considered by way of example. Hence, herein a plurality of radiotherapy treatment procedures take place distributed over the overall period of several years at respective time points, that, for example, may be several months or even several years apart.

At a time point of a first treatment procedure, initially referred to as the first time point, in act S1, a first partial model of the virtual patient model of the pediatric patient, that describes at least the surface thereof in three dimensions, but may optionally also describe further anatomical features and/or organs, is created. This may, for example, take place on the basis of image data of the pediatric patient or by adapting a universal model on the basis of first patient parameters describing the patient at the first time point. In the present case, these patient parameters include height, weight, age and gender, wherein, here, for the sake of simplicity, gender is assumed to be unchangeable. To generate the initially still three-dimensional virtual patient model, i.e., the first partial model, it is also possible to use a combination of the aforementioned procedures; ultimately, it is possible to use any procedures known.

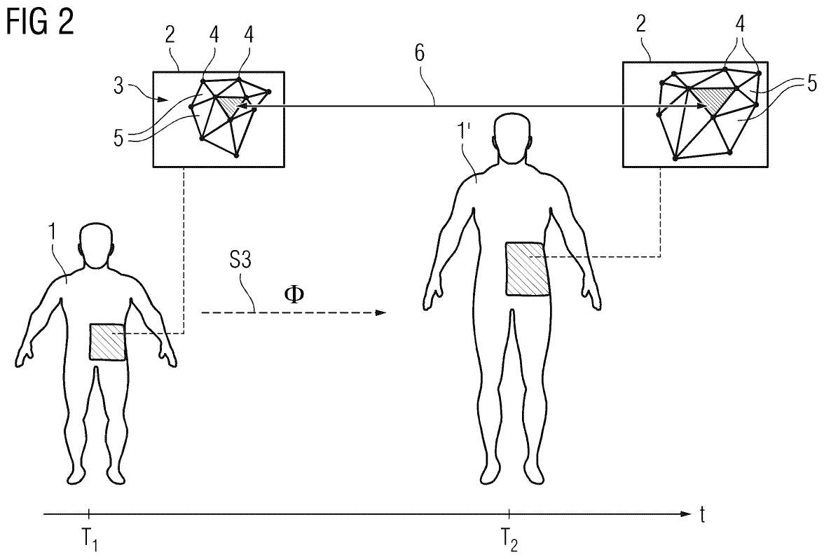

FIG. 2 depicts by way of example a schematic view of the three-dimensional virtual partial model 1 of the patient at the first time point T₁, that, therefore, describes the patient, for example the patient's surface, in three dimensions at the first time point T₁ with the corresponding first patient parameters. Herein, as shown in the enlarged region 2, in the present case, the surface is modeled as a polygonal model 3, i.e., by a mesh with vertices 4 and faces 5 spanned thereby. At a later second time point T₂, a further treatment procedure now takes place in the context of the irradiation treatment. To return to FIG. 1, in act S2, during this further treatment procedure, second current patient parameters of the patient are recorded, i.e., once again height, weight and age, since the patient's body has changed due to the long time difference, here due to growth. In order nevertheless to track the spatial position of spatial features (locations), for example vertices 4 and faces 5, in act S3, the virtual patient model is extended to the second time point T₂ by model transformation Φ, that, in the present case, only depends on the first and second patient parameters. To return to FIG. 2, this means that, by the model transformation Φ, a three-dimensional virtual partial model 1′ of the patient describing at least the surface of the patient at the second time point T₂ is ascertained from the three-dimensional virtual partial model 1 at the first time point T₁ by changing the position of the vertices 4 while retaining the respective neighborhoods and identities. Overall, therefore, a four-dimensional virtual patient model 1, 1′ is created since the surface of the patient is described by the three-dimensional virtual partial models 1 and 1′ at different time points T₁, T₂. Since the model transformation Φ only changes the position of the vertices 4, but not their neighborhood relationships or number, i.e., the identity is preserved, these as well as faces 5 may be uniquely assigned to one another at the different time points T₁, T₂, as is indicated for the dashed face 5 and the assignment arrow 6 in FIG. 2.

Herein, in the present case, the model transformation Φ was ascertained statistically by performing principal component analyses for scan datasets with assigned patient parameters for a population to be ascertained and using known methods to derive the model transformation Φ from the results and the assigned patient parameters. Other options for deriving the model transformation Φ may be used, for example using machine learning/artificial intelligence.

In act S1, an impact on the body caused by the first treatment procedure was noted in the virtual patient model 1 by assigning an item of impact information, here an item of dose information describing the skin dose, to an item of location information describing the spatial feature at which the impact occurred. Herein, impact information may be assigned to both the vertices 4 and the faces 5, wherein, on assignment to vertices 4, interpolation and/or extrapolation may take place for the faces 5 based on the respective vertices 4. Therefore, both the vertices 4 and the faces 5 may form spatial features in the embodiment shown here.

Since the spatial features are still identifiable at the second time point T₂ due to the model transformation Φ, it is also possible for the impact information from the first treatment procedure at the first time point T₁ to be transferred into the three-dimensional virtual partial model 1′; in addition, in act S3, newly arising impacts are added as impact information, here dose information. The impact information assigned to the spatial features, for example the vertices 4 and/or the faces 5, is created as a temporal course of dose information for this spatial feature of the four-dimensional virtual patient model 1, 1′.

In an optional act S4, representations may, for example, be derived from the four-dimensional virtual patient model 1, 1′ extended in this way that also reflect the impact information, here the administered radiation dose, and hence may also form a four-dimensional dose map, for example an X-ray dose map as a time series. Other uses of the extended four-dimensional virtual patient model 1, 1′ are also conceivable, for example for planning the treatment procedure, as a positioning aid, as a basis for determining treatment parameters and the like.

Herein, the extension described here, for example always starting from time point T₁ of the first treatment procedure, may also take place for further treatment procedures, wherein a later previously second time point may of course be used as a new first time point. This continuous extension for further treatment procedures or other relevant time points is indicated by act S5 by checking whether the radiation treatment is now completed, i.e., its overall period is finished, or whether there are further relevant time points or time points of treatment procedures for which the four-dimensional virtual patient model 1, 1′ is to be extended so that it is then possible to return to act S2. Otherwise, the four-dimensional virtual patient model 1, 1′ may be archived together with the impact information in act S6, for example.

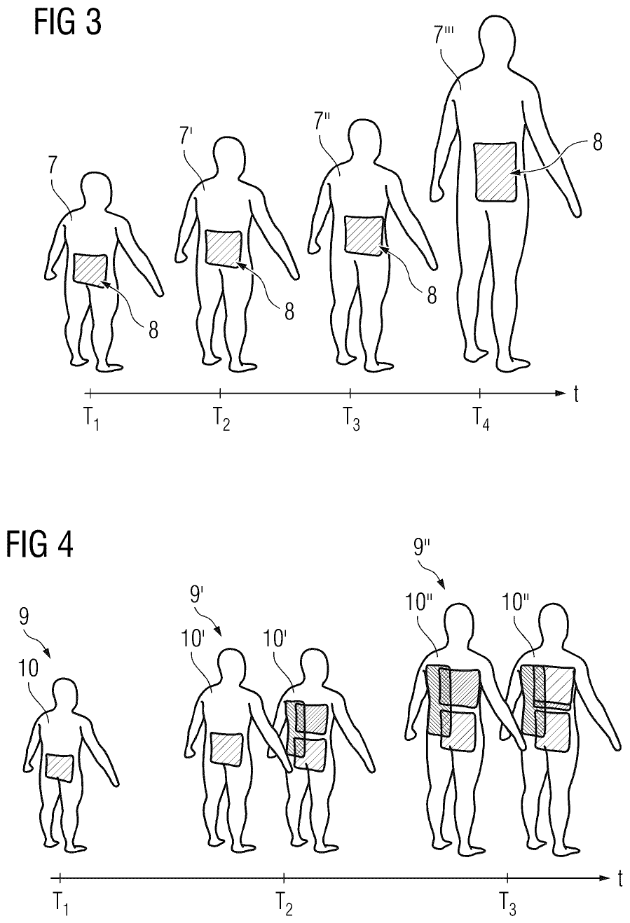

FIG. 3 explains by way of example this act-by-act further temporal addition to the four-dimensional virtual patient model, that in this figure is formed by the three-dimensional virtual partial models 7, 7′, 7″ and 7′″. Herein, the three-dimensional virtual partial models 7, 7′, 7″, 7′″ relate to time points T₁, T₂, T₃ and T₄. Assigned patient heights and patient weights may, for example, be 100 cm and 25 kg for time point T₁, 115 cm and 35 kg for time point T₂, 125 cm and 52 kg, for example, for time point T₃ and 175 cm and 75 kg, for example, for time point T₄. In addition to the body change illustrated here, the change in position and shape of an irradiated subregion 8 is also illustrated by way of example.

FIG. 4 depicts representations 9, 9′, 9″ in a further example. Here, not only is a three-dimensional representation of the respective partial model 10, 10′, 10″ possible—in the present case, this is color-coded with regard to irradiation procedures that have already takes place, as indicated by the hatched regions, wherein the situation before and after the treatment procedure is in each case illustrated for later treatment procedures (time points T₂ and T₃). Of course, new irradiated regions/dose information are added after the treatment procedure. The corresponding radiation dose is then summed up for the same spatial features.

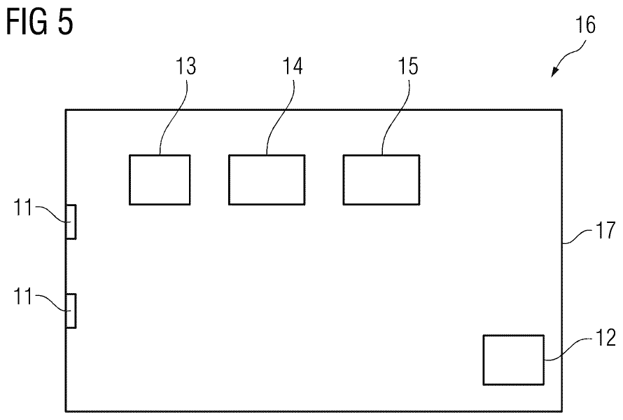

Finally, FIG. 5 depicts a functional schematic sketch of a patient model management facility 16, that in the present case includes a computing facility 17. The computing facility 17 has interfaces 11, shown by way of example, for outputting and receiving various types of data, for example patient parameters or representations 9, 9′, 9″, that may, for example, also be connected to input or output, not shown in greater detail here. A wide variety of data or information may be stored in a storage 12 in the short or long term; for example, it is possible, for example, for the four-dimensional virtual patient model 1, 1′ or 7, 7′, 7″, 7″ or 10, 10′, 10″ that develops over time to be held there.

The patient model management facility 16 is configured to perform the method and, for this purpose, initially has a creation unit 13 for performing act S1, i.e., for creating the first three-dimensional partial model (1, 7, 10). In an extension unit 14, the corresponding extension according to steps S2 and S3 may then take place at a second time point. Representations 9, 9′, 9″ according to act S4 may be generated in a representation unit 15. Of course, other further functional units are also conceivable in principle.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for managing a virtual patient model of a patient for a series of treatment, examination procedures, or treatment and examination procedures occurring over time, the method comprising:
describing, by the virtual patient model at least at a first time point at which the patient is described by first patient parameters, at least a surface of the patient at the first time point; and
extending the virtual patient model at least at a second later time point at which the patient is described by second patient parameters, using the first patient parameters and second patient parameters for describing at least the surface of the patient by at least one model transformation at the second time point.

2. The method of claim 1, wherein the patient model is at least partially a polygonal model with vertices and faces defined thereby, wherein the at least one model transformation changes a position of vertices based on the first patient parameters and the second patient parameters.

3. The method of claim 1, wherein spatial features of the patient model described by location information at the first time point are tracked at the second time point.

4. The method of claim 3, wherein for at least one time point, impact information related to the treatment, examination procedures, or treatment and examination procedures is assigned to the location information for creating a four-dimensional impact map based on the patient model.

5. The method of claim 4, wherein for radiation treatment, radiation examinations, or radiation treatment and radiation examinations the impact information comprises dose information.

6. The method of claim 4, characterized in that a representation of the patient is generated from the patient model for at least one of the time points covered thereby in which the impact information is reproduced true to location.

7. The method of claim 6, wherein the representation is of at least the patient's surface.

8. The method of claim 6, wherein the impact information is provided by color coding.

9. The method of claim 1, wherein the model transformation is ascertained statistically or by machine learning.

10. The method of claim 9, wherein for the statistical ascertainment of the model transformation, three-dimensional scan datasets of a population to be ascertained, that are provided and describe at least the surface of a person for different time points and to which in each case the patient parameters for the respective time points are assigned, are subjected to principal component analysis for classes of comparable patient parameters and/or at least substantially the same patient parameters and, to ascertain the model transformation, results of the principal component analysis are related to the assigned patient parameters.

11. The method of claim 1, characterized in that the first patient parameters and the second patient parameters comprise at least one of a patient's height, weight, age, or gender.

12. A patient model management facility for managing a virtual patient model of a patient for a series of treatment, examination procedures, or treatment and examination procedures occurring over time, wherein, at least at a first time point at which the patient is described by first patient parameters, the patient model describes at least a surface of the patient at the first time point, the patient model management facility comprising:
an extension unit for temporally extending the patient model for describing at least the surface of the patient at a second time point at which the patient is described by second patient parameters by at least one model transformation using the first patient parameters and the second patient parameters.

13. A non-transitory computer implemented storage medium that stores machine-readable instructions executable by at least one processor for managing a virtual patient model of a patient for a series of treatment, examination procedures, or treatment and examination procedures occurring over time, the machine-readable instructions comprising:
describing, by the virtual patient model at least at a first time point at which the patient is described by first patient parameters, at least a surface of the patient at the first time point; and
extending the virtual patient model at least at a second later time point at which the patient is described by second patient parameters, using the first patient parameters and second patient parameters for describing at least the surface of the patient by at least one model transformation at the second time point.

14. The non-transitory computer implemented storage medium of claim 13, wherein the patient model is at least partially a polygonal model with vertices and faces defined thereby, wherein the at least one model transformation changes a position of vertices based on the first patient parameters and the second patient parameters.

15. The non-transitory computer implemented storage medium of claim 13, wherein spatial features of the patient model described by location information at the first time point are tracked at the second time point.

16. The non-transitory computer implemented storage medium of claim 15, wherein for at least one time point, impact information related to the treatment, examination procedures, or treatment and examination procedures is assigned to the location information for creating a four-dimensional impact map based on the patient model.

17. The non-transitory computer implemented storage medium of claim 16, wherein for radiation treatment, radiation examinations, or radiation treatment and radiation examinations the impact information comprises dose information.

18. The non-transitory computer implemented storage medium of claim 16, characterized in that a representation of the patient is generated from the patient model for at least one of the time points covered thereby in which the impact information is reproduced true to location.

19. The non-transitory computer implemented storage medium of claim 18, wherein the representation is of at least the patient's surface.

20. The non-transitory computer implemented storage medium of claim 18, wherein the impact information is provided by color coding.
